(19) United States
(12) Patent Application Publication
(10) Pub. No.: US 2014/0074054 A1
(43) Pub. Date: Mar. 13, 2014

(54) Phototherapy Diaper

(71) Applicant: Natalie Lorene Davenport, Neosho, MO (US)

(72) Inventor: Natalie Lorene Davenport, Neosho, MO (US)

(21) Appl. No.: 13/607,453

(22) Filed: Sep. 7, 2012

(51) Int. Cl. A61N 5/06; A61F 13/62

(52) U.S. Cl. CPC A61N 5/0621; A61F 13/62
USPC 604/361; 604/385.01

(57) ABSTRACT

A versatile containment system and apparatus for enclosing urine, fecal matter, bodily excretions, which additionally acts as a disposable phototherapy diaper intended primarily to maximize the surface area of an infant's skin available to light in order to assist in phototherapy, while protecting individuals rights to privacy, is presented. The system additionally includes versatile affixing mechanisms.

### FIELD OF THE INVENTION

The instant embodiment generally relates to diapers, and more particularly, to diapers intended to increase the amount of skin surface area available to phototherapy light to aid in conditions such as hyperbilirubinemia.

### BACKGROUND OF THE INVENTION

Diapers that maximize the amount of available skin surface area are useful in the treatment of certain conditions. For example, phototherapy diapers can be useful in the treatment of infant jaundice. Bilirubin is a yellow pigment that is formed as the body removes old red blood cells. The pigment is broken down by the liver into smaller components, which are removed by the body in the stool. Often, infants are unable to efficiently break down bilirubin, resulting in a condition called hyperbilirubinemia that causes the skin and eyes to turn a yellowish color. Typical treatment involves phototherapy, which is a process of exposing the skin to ultraviolet light to increase the rate of bilirubin degradation. Unfortunately, the amount of skin available to phototherapy lights is limited by the infant's diaper. This has led to the creation of special phototherapy diapers intended to maximize the amount of exposed skin. The current state of the art in phototherapy diapers includes designs that increase the amount of surface area available to phototherapy light by using a light-permeable material.

### SUMMARY OF THE INVENTION

The instant apparatus and system, as illustrated herein, is clearly not anticipated, rendered obvious, or even present in any of the prior art mechanisms, either alone or in any combination thereof. A versatile containment system for urine, fecal matter, bodily excretions, which additionally acts as a disposable phototherapy diaper intended primarily to maximize the surface area of the wearer's skin available to light in order to assist in phototherapy is presented, while providing adequate genital coverage which protects the rights of the individual. Thus the several embodiments of the instant apparatus are illustrated herein.

It is therefore an object of the present system, in one example, to provide a diaper that maximizes the amount of light-available surface area to promote bilirubin degradation during phototherapy.

It another object of the apparatus to improve the effectiveness of phototherapy treatment, thereby decreasing hospital and insurance costs, promoting the wellness of the individual, and generating longer bonding times with benefits to the whole family.

It is yet another object of the apparatus to adequately cover the genital region of the wearer, promoting the general right to privacy.

It is yet another object of the present apparatus to provide a diaper that fits the wearer securely and includes a pocket for the storage of bodily wastes.

It is a further object of the present apparatus to provide an indicator of when the wearer has had a bodily excretion to aid nursing staff in determining when to change the diaper.

The invention results, in one example, from the realization that the problems of insufficient surface area, containment of infant waste, and wasted hospital staff labor are solved by a phototherapy diaper that uses less material by having one narrower end, forms an internal pocket through the use of an elastic band, and indicates the presence of a bowel movement with an external indicator.

The instant apparatus features a sheet of light permeable and flexible material that includes a set of broad ends and a set of narrow ends, each comprising a left lateral end and a right lateral end and wherein the sheet maximizes the surface area of an infant's skin available to light. The apparatus includes a fastening mechanism for affixing the broad and narrow ends utilizing the left lateral end and right lateral end. The apparatus also features an absorbent pad positioned in the center of the diaper, wherein the absorbent pad is disposed to line a sufficient area of a user to absorb urine and hold stool excretions. The fastening mechanism may be comprised of an adhesive or Velcro tab affixed to the left lateral end and right lateral ends of the broad end. The apparatus may feature a visual indicator built into the absorbent pad, as well as an elongated elastic means extending longitudinally along, but spaced apart from, the left and right lateral edges. The absorbent pad may be of an elliptical or rectangular shape.

There has thus been outlined, rather broadly, the more important features of the phototherapy diaper embodiments in order that the detailed description thereof that follows may be better understood, and in order that the present contribution to the art may be better appreciated. There are additional features of the apparatus that will be described hereinafter and which will form the subject matter of the claims appended hereto.

In this respect, before explaining at least one embodiment of the apparatus in detail, it is to be understood that the apparatus is not limited in its application to the details of construction and to the arrangements of the components set forth in the following description or illustrated in the drawings. The apparatus is capable of other embodiments and of being practiced and carried out in various ways. Also, it is to be understood that the phraseology and terminology employed herein are for the purpose of description and should not be regarded as limiting.

These together with other objects of the apparatus, along with the various features of novelty, which characterize the apparatus, are pointed out with particularity in the claims annexed to and forming a part of this disclosure. For a better understanding of the apparatus, its operating advantages and the specific objects attained by its uses, reference should be made to the accompanying drawings and descriptive matter in which there are illustrated preferred embodiments of the apparatus.

To the accomplishment of the foregoing and related ends, certain illustrative aspects are described herein and these aspects are indicative of the various ways in which the principles disclosed herein can be practiced and all aspects and equivalents thereof are intended to be within the scope of the claimed subject matter.

### BRIEF DESCRIPTION OF THE DRAWINGS

Advantages of the present apparatus will be apparent from the following detailed description of exemplary embodiments thereof, which description should be considered in conjunction with the accompanying drawings, in which: Having thus described the invention in general terms, reference will now be made to the accompanying drawings, which are not necessarily drawn to scale, and wherein:

FIG. 1 depicts a front view of a diaper in accordance with the prior art.

FIG. 2 depicts a front view of a phototherapy diaper in accordance with the prior art.

FIG. 3 depicts a front view of an embodiment of the instant invention.

FIG. 4 depicts a front view of an embodiment of the instant invention that includes a visual indicator.

FIG. 5 depicts a focused front view of an infant wearing an example of the instant invention.

### DETAILED DESCRIPTION OF THE SEVERAL EMBODIMENTS

The detailed description set forth below in connection with the appended drawings is intended as a description of presently preferred embodiments of the invention and does not represent the only forms in which the present invention may be constructed and/or utilized. The description sets forth the functions and the sequence of steps for constructing and operating the invention in connection with the illustrated embodiments.

Phototherapy diapers are useful in the treatment of several medical conditions, including hyperbilirubinemia, a condition that causes yellowing of the skin, membranes, and eyes. Hyperbilirubinemia results from a build-up of bilirubin generated by the removal of old blood cells. Newborn infants are more likely to develop hyperbilirubinemia from having under-developed livers, consuming proteins in breast milk that block bilirubin degradation, and other factors. Hyperbilirubinemia strikes 10-15% of newborns and is more common in premature infants.

The condition is typically treated by phototherapy. Exposing skin to ultraviolet light helps the body break down bilirubin for disposal through the stool. Research has shown that naked infants have better results with phototherapy than those that wear diapers. However, diapers are often necessary in a hospital environment as they facilitate the fast cleaning of infant waste, promote sanitary facilities, and protect the individual patients' right to privacy with adequate coverage of the genital region.

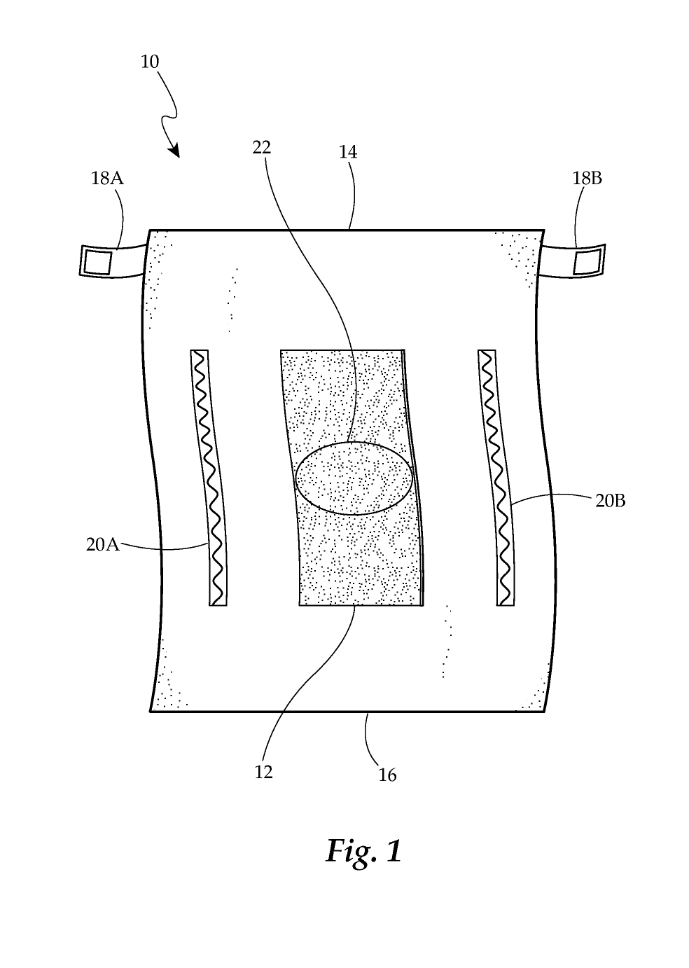

FIG. 1 illustrates a diaper apparatus 10 in accordance with the prior art. An absorbent pad 12 is affixed to a flexible sheet 11 with top end 14 and bottom end 16 of equal size. Top end 14 has a left adhesive tab 18A and right adhesive tab 18B. When folded over, elastic bands 20A and 20B give the diaper a curved structure to both fit comfortably around the infant and also create a pocket 22 to contain infant wastes.

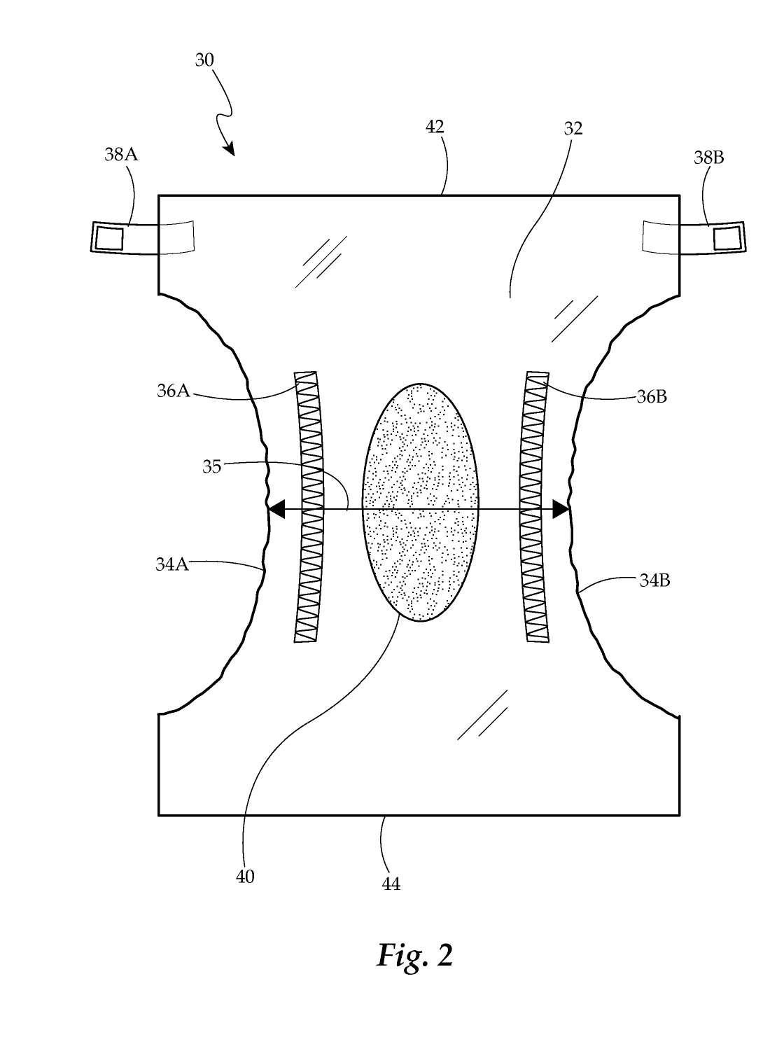

Diapers composed of a light-permeable material increase the amount of skin surface area exposed to ultraviolet light. FIG. 2 illustrates an improved diaper 30 for use in phototherapy. Using a light-permeable sheet 32 allows some light exposure to the region covered by the diaper. Left edge 34A and right edge 34B taper inwards from top end 42 and bottom end 44, creating a laterally narrowed region 35 that reduces the amount of material used in the sheet and also increases the level of skin surface area exposed. The absorbent pad 40 is slightly smaller, allowing more light to penetrate the diaper. Similar to the diaper in FIG. 1, elastic bands are incorporated that cause the diaper to curve when folded. Adhesive tabs 38A and 38B on top end 42 can be affixed to bottom end 44 when the diaper is folded to secure the diaper to the infant.

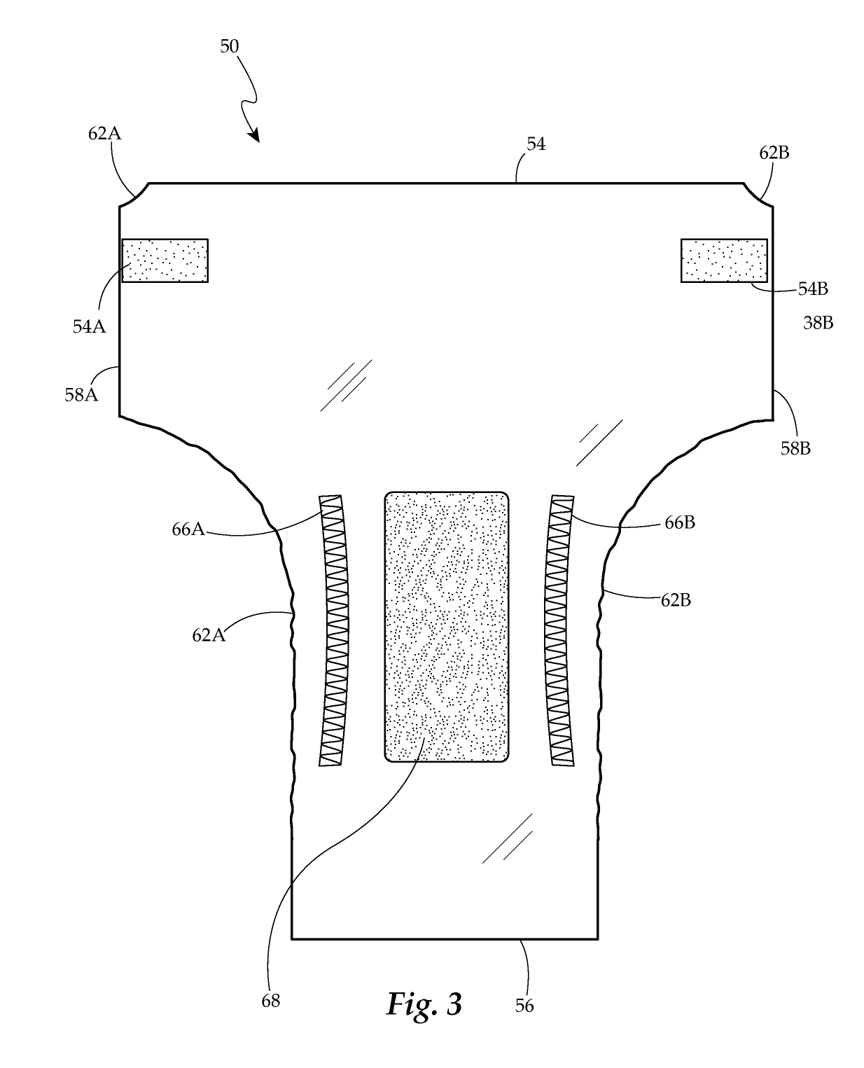

The ideal phototherapy diaper will be composed of a design that both maximizes uncovered skin surface area and minimizes the amount of skin that is covered, even by a light-permeable material, while simultaneously protecting the individual's rights. Adopting a diaper design that includes only two tabbed edges and a narrower bottom end increases the amount of skin surface area available for direct phototherapy light. FIG. 3 illustrates an embodiment of the instant invention 50, wherein a light-permeable sheet 52 has a broader top end 54 and a narrower bottom end 56. Left end 58A and right end 58B have adhesive tabs 54A and 54B. Left end 58A and right end 58B have tapered edges 62A and 62B that meet with the broader top end, and tapered edges 60A and 60B that meet with the bottom end. An absorbent pad 68 is affixed to the center of the light-permeable sheet 52. Elastic bands 66A and 66B constrict the sheet, pulling it inwards to create a curvature when folded. The diaper is secured to the infant by folding the diaper such that adhesive tabs 54A and 54B are affixed to bottom end 56. This results in a secure fit that makes the diaper easy to adjust and remove.

The addition of elastic bands 66A and 66B spaced apart from the lateral edges 58A and 58B causes the diaper 56 to form a pocket 59 when worn, allowing sufficient room for infant wastes. The pocket 59 allows for further light exposure by providing space between the skin and the absorbent pad, allowing light to scatter and cover more skin. Additionally, the elastic bands 66A and 66B cause the tapered edges of the diaper to conform to the infant's legs, maximizing the amount of leg surface area that is available for direct exposure to phototherapy.

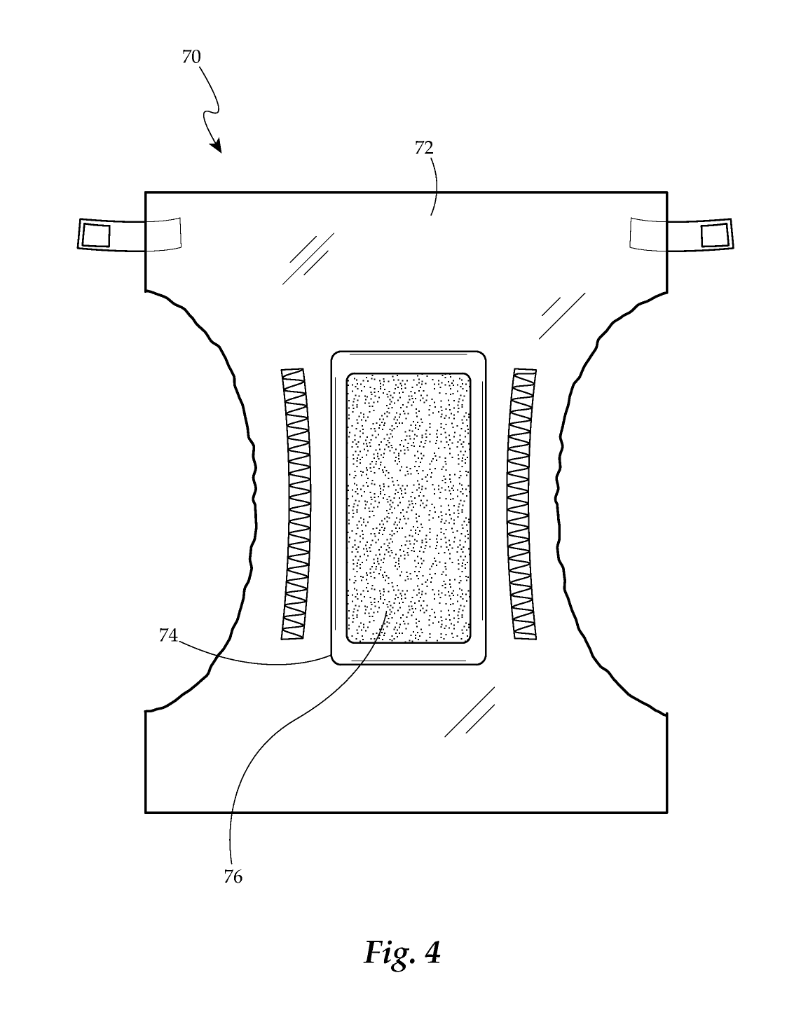

The addition of a visual indicator to alert hospital staff that a diaper requires changing will result in diapers changed less frequently and more efficiently, ultimately leading to increased surface area available for phototherapy, less wasted time changing diapers, and more bonding time for the wearer and family. FIG. 4 illustrates an example in accordance with the instant invention. Absorbent pad 74 is attached to light-permeable sheet 72. Absorbent pad 74 includes visual indicator 76 that changes color when it is exposed to infant wastes. Diapers with a visual indicator of when an infant has excreted can facilitate a faster and more efficient diaper changing schedule. Quickly removing waste and applying a clean diaper will prevent the waste from blocking phototherapy light for an extended period of time. The visual indicator 76 has the added benefit of reducing hospital staff or caregiver time, as only diapers that contain waste will be changed.

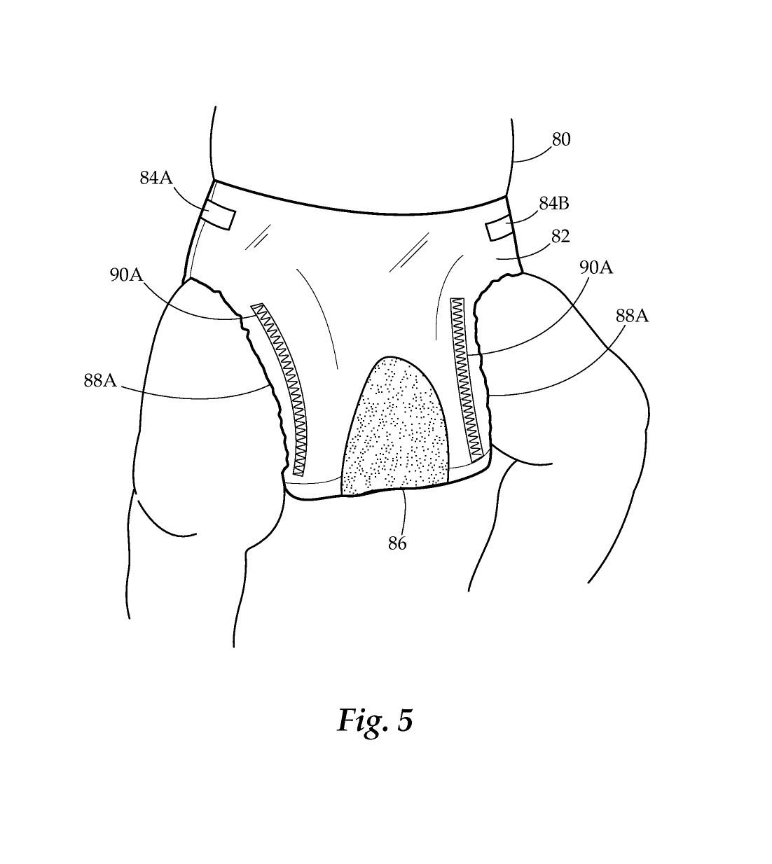

FIG. 5 illustrates an infant 80 wearing an example of the instant invention. The diaper 82 conforms snugly to the infant, attached with adhesive tabs 84A and 84B. Elastic bands 90A and 90B on the inner surface of the diaper cause edges 88A and 88B to conform to the shape of the infant's legs. Visual indicator 86 has changed color, indicating a current need to change the diaper. It should be noted that the diaper 82 can be used for patients of many ages, including those who have incontinence, who either need assistance with diaper changes or have decreased awareness. While employing a design that maximizes the amount of phototherapy light available to the skin, the diaper 82 is still able to adequately cover the genital region, ensuring individual privacy. The visual indicator helps the caregiver in noticing a needed change of the diaper.

What is claimed is:

1. A disposable phototherapy diaper comprising:
a sheet of light-permeable and flexible material comprising a set of broad ends and a set of narrow ends each comprising a left lateral end and a right lateral end and wherein the sheet maximizes the surface area of an infant's skin available to light;
a fastening mechanism for affixing the broad ends to the narrow ends utilizing the left lateral end and right lateral end; and,
an absorbent pad that is positioned in the center of the diaper, wherein the absorbent pad is disposed to line a sufficient area of a user to absorb urine and hold stool excretions.

2. The disposable phototherapy diaper of claim 1, wherein the fastening mechanism comprises an adhesive tab affixed to the left lateral and right lateral ends of the broad end.

3. The disposable phototherapy diaper of claim 1, where the fastening means is a Velcro tab affixed to the left and right lateral ends of the broad end.

4. The disposable phototherapy diaper of claim 1, where the left and right lateral ends of the broad end include at least one left and at least one right tab.

5. The disposable phototherapy diaper of claim 4, where the fastening means includes an adhesive tab attached to the left and right tabs.

6. The disposable phototherapy diaper of claim 4, where the fastening means includes a Velcro tab attached to the left and right tabs.

7. The disposable phototherapy diaper of claim 1, where the left and right lateral ends of the narrow end include left and right tabs.

8. The disposable phototherapy diaper of claim 7, where the fastening means includes an adhesive tab attached to the left and right tabs.

9. The disposable phototherapy diaper of claim 7, where the fastening means includes a Velcro tab attached to the left and right tabs.

10. The disposable phototherapy diaper of claim 1, including a visual indicator of when the infant has had a bodily excretion to indicate that the diaper requires changing.

11. The disposable phototherapy diaper of claim 10, where the visual indicator is built into the absorbent pad.

12. The disposable phototherapy diaper of claim 1, including an elongated elastic means extending longitudinally along, but spaced apart from, the left and right lateral edges.

13. The disposable phototherapy diaper of claim 1, where the absorbent pad is in a rectangular shape.

14. The disposable phototherapy diaper of claim 1, where the absorbent pad is in an elliptical shape.

15. A disposable phototherapy diaper intended primarily to maximize the surface area of an infant's skin available to light, comprising:
a sheet of light-permeable flexible material extending longitudinally between a broad end and a narrow end, wherein each of the broad end and the narrow end each comprise a left lateral end and a right lateral end, wherein the left lateral end and the right lateral end of the broad end comprise a left tab and a right tabs and wherein the sheet of light-permeable flexible material is oriented in two planes prior to use;
a fastening mechanism disposed to join the left and right tabs to the narrow end upon affixing of the diaper is to a user; and,
a pad of absorbent material having a size strictly coextensive with the crotch of the infant to receive and retain bodily wastes.

16. The diaper of claim 15, wherein the left lateral end and the right lateral end are curved as they taper longitudinally from the broad end to the narrow end.
